(19) United States
(12) Patent Application Publication
(10) Pub. No.: US 2026/0041387 A1
(43) Pub. Date: Feb. 12, 2026

(54) Radiation Detection Device and Medical Imaging Equipment

(71) Applicant: Shanghai United Imaging Healthcare Co., Ltd., Shanghai (CN)

(72) Inventors: Chengzhe Zhang, Shanghai (CN); Junwen Lin, Shanghai (CN); Guoxuan Qin, Shanghai (CN); Guangbiao Chen, Shanghai (CN)

(21) Appl. No.: 19/302,269

(22) Filed: Aug. 18, 2025

Related U.S. Application Data
(63) Continuation of application No. PCT/CN2024/076986, filed on Feb. 8, 2024.

(30) Foreign Application Priority Data
Feb. 16, 2023 (CN) 202310128759.7

(51) Int. Cl. A61B 6/00

(52) U.S. Cl. CPC A61B 6/4476; A61B 6/4441; A61B 6/4447

(57) ABSTRACT

A radiation detection device and medical imaging equipment. The radiation detection device includes a lifting module. The lifting module includes a base, a first lifting unit, and a second lifting unit. The first lifting unit includes a first lifting component extending along a Z-axis, which is movably connected to the base along the Z-axis. The second lifting unit includes a driving component extending along the Z-axis and a second lifting component connected to the driving component, which is configured to drive the second lifting component to move along the Z-axis. The direction of the Z-axis is perpendicular to the connection surface of the base, wherein the connection surface of the base is the surface of the base connected to the first lifting component and the driving component.

### CROSS-REFERENCE TO RELATED APPLICATIONS

This application is a continuation of International Application No. PCT/CN2024/076986, filed on Feb. 8, 2024, which claims priority to Chinese Patent Application No. 202310128759.7, filed on Feb. 16, 2023, titled “Radiation Detection Device and Medical Imaging Equipment”, the entire contents of each of which are hereby incorporated by reference.

### TECHNICAL FIELD

The present disclosure relates to the technical field of medical devices, particularly to a radiation detection device and a medical imaging equipment.

### BACKGROUND

With the improvement of medical standards, the use of angiography machines in hospitals has become increasingly common. Angiography machines are used in diagnostic and interventional surgeries in major hospitals. When diagnosing or performing interventional surgery, the detector needs to move up and down to change the range of the imaging area, so a lifting module is needed to achieve the up and down movement of the detector. Specifically, the lifting module is configured to ensure that the detector moves up and down within a certain range, thereby adjusting the distance between the radiation source of the angiography machine and the imaging plane of the detector (referred to as source-imaging distance, SID).

Typically, the detector of the angiography machine is installed on the installation plate at the bottom of the lifting module, and the lifting module enables the detector to move up and down in a straight line to change the range of the imaging area. At present, the lifting modules of detectors on the market mainly use single-stage lifting modules with ball screws, rack, and pinion, or double-stage lifting modules with multiple screws nested. The single-stage lifting modules take up a lot of space in the lifting direction, and the related guiding structures (such as rails) are exposed, which can affect the movement of the C-arm and pose a safety hazard. When a two-stage lifting module with multiple lead screws nested is used, the relative motion along a straight line between each level of the lifting module during the lifting movement of the detector is uncertain, affecting the stability of the lifting movement of the detector.

In addition, the single-stage lifting module is typically installed on one end of the arc direction of the C-arm of the angiography machine. To meet the total arc length requirement of the C-arm, the arc length at the other end of the C-arm needs to be adaptively extended, causing the other end of the C-arm to protrude outward, which can have a certain impact on the movement of the C-arm and easily cause safety hazards.

Therefore, it is necessary to provide a new lifting module that reduces the space occupied by the detector and the lifting module as a whole in the lift direction while the lift range of the detector keeps unchanged, and ensures that the C-arm cooperating with the new lifting module does not need to extend the arc length of the end without the detector installed.

### SUMMARY

With the aim of reducing the space occupied by the detector and the lifting module as a whole in the lifting direction while the lifting range of the detector keeps unchanged, the present disclosure provides a radiation detection device and medical imaging equipment.

To solve the above technical problem, based on one aspect of the present disclosure, a radiation detection device is provided. The radiation detection device includes a lifting module. The lifting module includes a base, a first lifting unit, and a second lifting unit. The first lifting unit includes a first lifting component extending along a Z-axis. The first lifting component is movably connected to the base along the Z-axis. The second lifting unit includes a driving component extending along the Z-axis and a second lifting component connected to the driving component The driving component is configured to drive the second lifting component to move along the Z-axis. A direction of the Z-axis is perpendicular to a connection surface of the base (20). The connection surface of the base is a surface on which the base is connected to the first lifting component and the driving component.

In one of the embodiments, the radiation detection device further includes a detector and at least two installation bodies arranged along the Z-axis. One of at least two adjacent installation bodies of the at least two installation bodies are connected to the first lifting component and another one of the at least two adjacent installation bodies is connected to the second lifting component. The detector is connected to any one of the at least two adjacent installation bodies of the at least two installation bodies. For example, the detector is connected to the one of the at least two adjacent installation bodies that is connected to the first lifting component. As another example, the detector is connected to the another one of the at least two adjacent installation bodies that is connected to the second lifting component. In some embodiments, the at least two adjacent installation bodies include two adjacent installation bodies. One of the at least two adjacent installation bodies is connected to the first lifting component, and the other one of the at least two adjacent installation bodies is connected to the second lifting component.

In one of the embodiments, the second lifting component and the driving component are eccentrically arranged relative to the z-axis. A section of the second lifting component perpendicular to the z-axis is partially overlapped with a section of the driving component perpendicular to the z-axis.

In one of the embodiments, the first lifting unit further includes a reference component connected to the first lifting component. The reference component is configured to be rotatable around its own axis. The first lifting unit is configured to convert a rotational motion of the reference component into a linear motion of the first lifting component along the z-axis.

In one of the embodiments, at least one segment of the first lifting component is configured as a first lead screw, and the reference component is threaded to the first lead screw. Alternatively, at least one segment of the first lifting component is configured as a rack, and the reference component meshes with the rack.

In one of the embodiments, the driving component is configured to be rotatably connected with the base around the z-axis. A rotation of the driving component drives the second lifting component to move along the Z-axis.

In one of the embodiments, the lifting module further includes a driving unit. The driving unit is respectively connected to the reference component and the driving component. The driving unit is configured to drive the reference component and the driving component to rotate synchronously.

In one of the embodiments, the driving unit includes a first transmission structure, a second transmission structure, a first gear, and a second gear. The first gear and the second gear are coaxially fixed along the Z-axis and can rotate synchronously around the Z-axis. One of the first gear and the second gear meshes with the first transmission structure. The first transmission structure meshes with the driving component. Another one of the first gear and the second gear meshes with the second transmission structure. The second transmission structure meshes with the reference component.

In one of the embodiments, at least one segment of the driving component is configured as a second lead screw, and the second lifting component is threaded with the second lead screw.

In one of the embodiments, moving rates of the first lifting component and the second lifting component are equal.

In one of the embodiments, the radiation detection device further includes an intermediate body. The intermediate body and one of the at least two installation bodies each have a cylindrical or shell-like shape around the z-axis. The base is fixed within the intermediate body. The intermediate body is movably lapped with at least two adjacent installation bodies of the at least two installation bodies along the Z-axis respectively. The intermediate body is in conjunction with the at least two adjacent installation bodies to cover the lifting module. In some embodiments, the at least two adjacent installation bodies are the at least two adjacent installation bodies one of which is connected to the first lifting component and another one of which is connected to the second lifting component described above.

In one of the embodiments, the first lifting component is provided with a connecting component. The first lifting component is connected to one of the at least two adjacent installation bodies through the connecting component.

In one of the embodiments, the second lifting component and the driving component are concentric with respect to the Z-axis. The driving component has a tubular structure. The driving component is configured to be movably inserted through the second lifting component.

In one of the embodiments, the second lifting component includes a first part and a second part connected to each other. The first part is mechanically connected to the driving component by thread. The second part is mechanically connected to the driving component. The second part is connected to another one of the at least two adjacent installation bodies. The first part is configured to be driven by the driving component to move along the Z-axis.

In one of the embodiments, the first transmission structure and the second transmission structure are synchronous transmission belts or are a combination of multiple gear parts.

In one of the embodiments, the driving unit further includes a rotating motor, a coupling, and an installation shaft. An axial direction of the installation shaft is the same as a direction of the Z-axis. The rotating motor is set on the base through a motor installation seat. The installation shaft is connected to an output shaft of the rotating motor through the coupling. The first gear and the second gear are coaxially fixed on the installation shaft.

In one of the embodiments, a guiding structure is provided between the intermediate body and at least part of the at least two installation bodies. For example, the at least part of the at least two installation bodies includes all of the at least two installation bodies. As another example, the at least part of the at least two installation bodies includes a part of the at least two installation bodies.

In one of the embodiments, the guiding structure includes a guiding groove, a guiding plate, or a guiding rail.

In one of the embodiments, at least one segment of the first lifting component is configured as a first lead screw The reference component is a nut. At least one segment of the first lifting component is configured as a rack. The reference component is configured as a gear.

In one of the embodiments, the first lifting component and the second lifting component move in opposite directions relative to the base.

A medical imaging equipment includes a C-arm and a radiation source. The C-arm has two ends opposite each other. Each of the two ends of the C-arm have an inner arc surface. The radiation source is installed on an inner arc surface at one end of the C-arm. In the radiation detection device, another one of the at least two adjacent installation bodies not connected to the detector is connected to an inner arc surface of another end of the C-arm.

In one of the embodiments, a direction of the Z-axis is approximately a direction of a line connecting the two ends of the C-arm.

A medical imaging equipment includes a gantry, a radiation source, and a radiation detection device. The gantry having two ends opposite each other. The radiation source is configured at one end of the gantry. The radiation detection device includes a lifting module and a detector. The lifting module includes a first lifting unit and a second lifting unit. The detector is arranged at another end of the gantry through the lifting module. The first lifting unit and the second lifting unit are arranged perpendicularly to the detector in parallel. The first lifting unit and the second lifting unit are configured to drive the detector to move near or away from the radiation source.

In one of the embodiments, the gantry is a C-arm having two ends opposite each other. Both ends of the C-arm have an inner arc surface. The radiation source is arranged on the inner arc surface at one end of the C-arm. The detector is arranged on the inner arc surface of the other end of the C-arm through the lifting module.

In one of the embodiments, the lifting module further includes a base. The first lifting unit includes a first lifting component extending along a Z-axis. The first lifting component is movably connected to the base along the Z-axis. The Z-axis is parallel to the direction of movement of the detector. The second lifting unit includes a driving component extending along the Z-axis. A second lifting component connected to the driving component. The driving component is configured to drive the second lifting component to move along the Z-axis.

In one of the embodiments, the medical imaging equipment further includes at least two installation bodies arranged along the Z-axis. One of at least two adjacent installation bodies of the at least two installation bodies are connected to the first lifting component, and another one of the at least two adjacent installation bodies is connected to the second lifting component. The detector is connected to one of the two adjacent installation bodies of the at least two installation bodies.

In one of the embodiments, the second lifting component and the driving component are eccentrically arranged relative to the z-axis. A section of the second lifting component perpendicular to the z-axis and a section of the driving component perpendicular to the z-axis partially overlap.

In one of the embodiments, the first lifting unit further includes a reference component connected to the first lifting component. The reference component is arranged to be rotatable around its own axis. The first lifting unit is configured to convert a rotational motion of the reference component into a linear motion of the first lifting component along the Z-axis.

In one of the embodiments, the driving component is configured to be rotatably connected with the base around the Z-axis. A rotation of the driving component drives the second lifting component to move along the Z-axis.

In one of the embodiments, at least one segment of the driving component is configured as a second lead screw. The second lifting component is threaded to the second lead screw.

In one of the embodiments, the first lifting component and the second lifting component move in opposite directions.

### BRIEF DESCRIPTION OF THE DRAWINGS

It should be understood by those skilled in the art that the drawings provided are intended for a better understanding of the present disclosure and do not constitute any limitation to the scope of the present disclosure. Wherein:

FIG. 1 is a schematic diagram of medical imaging equipment in the relevant technology;

FIG. 2 is a schematic diagram of a radiation detection device of one embodiment of the present disclosure;

FIG. 3 is a schematic diagram of a lifting module of one embodiment of the present disclosure;

FIG. 4 is a schematic diagram of a medical imaging equipment of one embodiment of the present disclosure;

FIG. 5 is an explosion diagram of a detector and installation body of one embodiment of the present disclosure;

FIG. 6 is a schematic diagram of an installation body of one embodiment of the present disclosure; and

FIG. 7 is a schematic diagram of an intermediate body of one embodiment of the present disclosure.

In the attached figure:
10—lifting structure;
20—Base;
30—first lifting unit; 31—first lifting component; 32—reference component;
40—second lifting unit; 41—second lifting component; 411—first part; 412—second part; 42—driving component;
50—driving unit; 51—first gear; 52—second gear; 53—third gear; 54—fourth gear; 55—rotating motor; 56—installation shaft; 57—coupling; 58—motor installation seat;
60—detector;
70—installation body; 71—connecting substrate; 72—installation substrate; 73—connecting component;
80—intermediate body;
90—C-arm;
100—radiation source.

### DETAILED DESCRIPTION

To make the purpose, advantages and features of the present disclosure clearer, further details of the application are provided below with reference to the drawings and specific embodiments. It should be noted that the drawings are in a very simplified form and are not drawn to scale, and are only intended to facilitate and clearly illustrate the purpose of the embodiments of the present disclosure. Furthermore, the structures shown in the drawings are often part of the actual structures. In particular, each drawing needs to show different focuses and sometimes uses different scales.

As used in this application, the singular forms “one”, “the” and “that” include plural objects. The term “or” is usually used with the meaning of “and/or”, and the term “several” is usually used with the meaning of “at least one”. The term “at least two” is usually used to include the meaning of “two or more”, and in addition, the terms “first”, “second”, “third” are used for descriptive purposes only and should not be understood as indicating or suggesting relative importance or implicitly indicating the number of technical features indicated. Thus, a feature that is defined as “first”, “second”, “third” may explicitly or implicitly include one or at least two of those features, and “one end” and “the other end” and “the near end” and “the far end” usually refer to the two corresponding parts, which not only include endpoints, but the terms “installation”, “connection”, “connection” should be understood in a broad sense, for example, It can be a fixed connection, or a detachable connection, or as a whole; It can be a mechanical connection or an electrical connection; It can be a direct connection or an indirect connection through an intermediate medium, it can be an internal connection between two components or an interaction relationship between two components. Furthermore, as used in the present disclosure, where one element is placed on another element, it usually only indicates that there is a connection, coupling, fit or transmission relationship between the two elements, and the two elements can be directly or indirectly connected, coupled, fit or transmitted through an intermediate element, and cannot be understood as indicating or suggesting a spatial position relationship between the two elements, That is, a component may be located in any position within, outside, above, below or on one side of the other component, unless otherwise expressly indicated in the content. For those skilled in the art, the specific meanings of the above terms in the present disclosure can be understood in accordance with the specific circumstances.

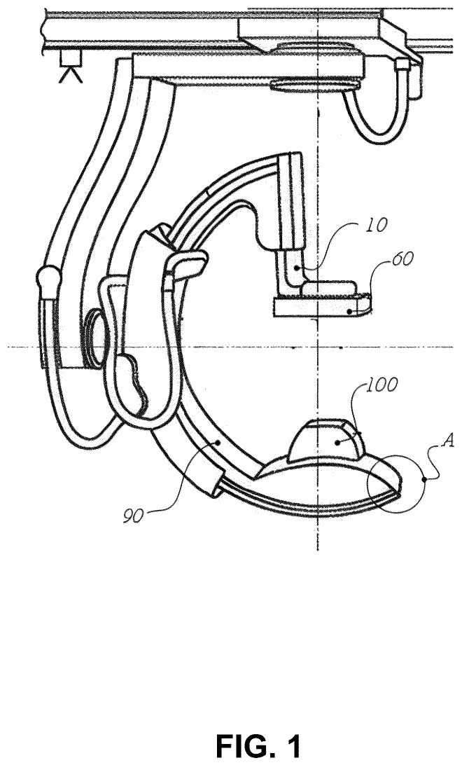

The medical imaging equipment is a Digital Subtraction Angiography (DSA) device, which can be used to assist in the fields of angiography, cardiology and neurology, which is not limited herein. FIG. 1 is a schematic diagram of a medical imaging equipment related to the technology. As shown in FIG. 1, a medical imaging equipment of the related technology is presented graphically, the medical imaging equipment includes a C-arm 90, a radiation source 100, a detector 60, and a lifting structure 10. The radiation source 100 is installed at one end of the C-arm 90, and the detector 60 is installed at the other end of the C-arm 90 through the lifting structure 10 so that the radiation source 100 and the detector 60 are aligned. It is understandable that a radiation source 100 is a device capable of emitting X-rays, gamma rays, or electron rays, and the detector 60 is a device capable of receiving the rays emitted by the radiation source 100. The combination of the radiation source 100 and the detector 60 enables medical examination or treatment, etc. In the related technology, the radiation source 100 includes a tube, which can emit rays (such as X-rays), and the detector 60 can be a flat plate detector 60. The rays emitted by the tube are received by the detector 60 after passing through the patient for imaging processing.

The mechanical features of the lifting structure 10 allow the detector 60 to move in the lift direction (here the lift direction can be understood as the direction of the line connecting the two ends of the C-arm 90), thus bringing the detector 60 closer to or away from the radiation source 100, thereby achieving that the relative distance between the focal point of the tube of the radiation source 100 and the imaging surface of the detector 60 (referred to as the Source-Imaging Distance, SID) is adjustable to accommodate a wider range of clinical diagnostics. In related technologies, lifting structures 10 are typically single-stage lift structures, such as those designed based on ball screws or rack and pinion, and there are also double-stage lifting structures 10 in some products, such as those designed based on bidirectional lead screws. Based on this, it can be known that the mechanical design of the lifting structure 10 applied to the detector 60 in the relevant technology results in the overall space occupied by the lifting structure 10 and the detector 60 in the lift direction being unadjustable, and in order to ensure a sufficiently large range of SID, it may also result in the overall space occupied by the lifting structure 10 and the detector 60 in the lift direction being relatively large.

In view of this, an embodiment of the present disclosure provides a radiation detection device for medical imaging equipment, with the aim of reducing the space occupied by the overall device in the lifting direction while the lifting motion range of the detector 60 remains unchanged.

In some embodiments, in the radiation detection device and the medical imaging equipment provided by the present disclosure, the radiation detection device includes a lifting module. The lifting module includes a base, a first lifting unit, and a second lifting unit. The first lifting unit includes a first lifting component extending along a Z-axis. The first lifting component is movably connected to the base along the Z-axis. The second lift unit includes a driving component extending along the Z-axis and a second lifting component connected to the driving component. The driving component is set to drive the second lifting component along the Z-axis.

Configured in this way, the first lifting component and the second lifting component of the lifting module extend linearly along the Z-axis. The first lifting component is movable along the Z-axis relative to the base. The driving component is stationary along the Z-axis relative to the base. The driving component drives the second lifting component to move along the Z-axis. By setting the first lifting component and second lifting component, the size of the entire lifting module along the Z-axis is adjustable, thereby reducing the space occupied by the radiation detection device along the Z-axis while the range of motion of the detector along the Z-axis remains unchanged. It should be noted that the medical imaging equipment of the present disclosure includes the radiation detection device and thus has the beneficial technical effect brought by the radiation detection device, which is not be repeated here. Moreover, the radiation source and the radiation detection device are respectively installed on inner arc surfaces at both ends of the C-arm, ensuring that the arc length of the end of the C-arm where the radiation source is installed does not need to be extended while the travel requirements of the C-arm slip ring movement are met, and ensuring the stability of the relative motion between the first lifting component and second lifting component, and ensuring the safety of the C-arm slip ring movement.

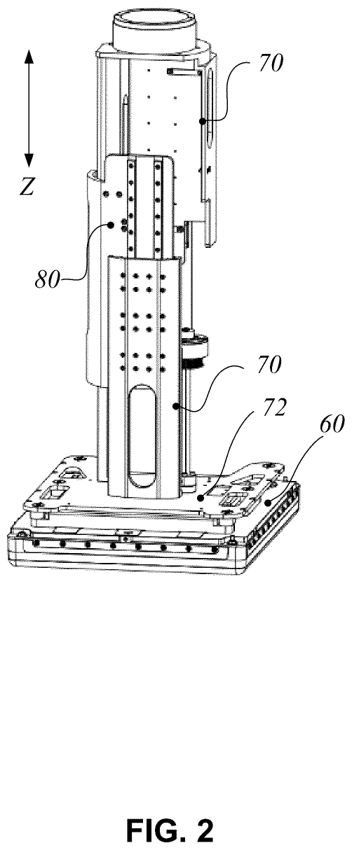

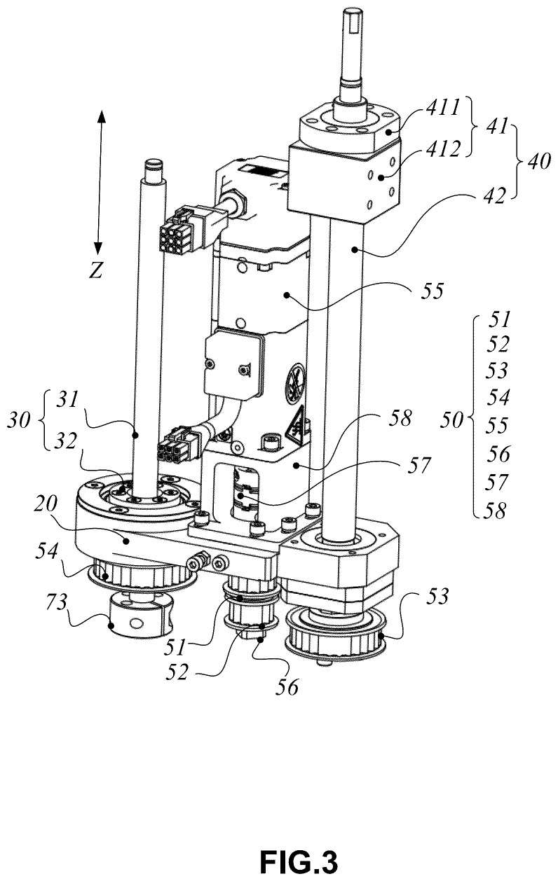

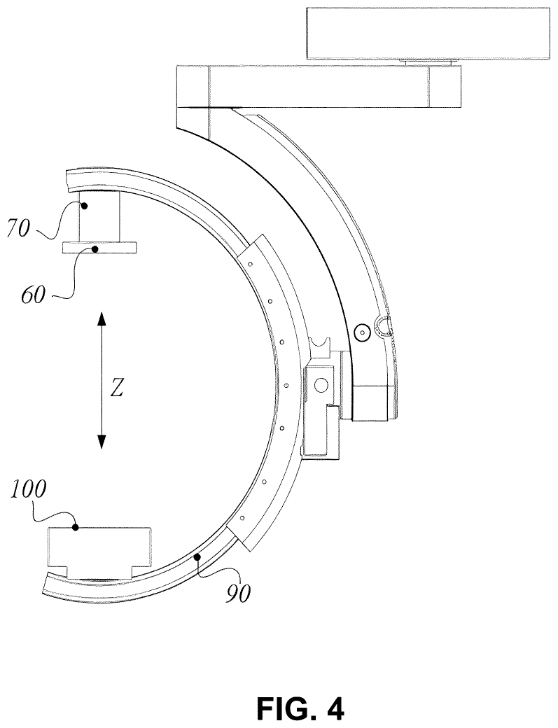

FIG. 2 is a schematic diagram of a radiation detection device of one embodiment of the present disclosure, FIG. 3 is a schematic diagram of a lifting module of one embodiment of the present disclosure, and FIG. 4 is a schematic diagram of a medical imaging equipment of one embodiment of the present disclosure. As shown in FIG. 2, the radiation detection device includes a lifting module. As shown in FIG. 3, the lifting module includes a base 20, a first lifting unit 30, and a second lifting unit 40. The first lifting unit 30 includes a first lifting component 31 extending along a Z-axis. The first lifting component 31 is movably connected to the base 20 along the Z-axis. The second lifting unit 40 includes a driving component 42 extending along the Z-axis and a second lifting component 41 connected to the driving component 42. The driving component 42 is configured to drive the second lifting component 41 to move along the Z-axis relative to the base 20. Thus, both the first lifting component 31 and the driving component 42 extend along the z-axis in a linear (e.g., rod-shaped) manner, and the first lifting component 31 is movable along the Z-axis relative to the base 20, the driving component 42 is stationary along the Z-axis relative to the base 20, and the second lifting component 41 is movable along the Z-axis relative to the base 20. For example, the direction of the Z-axis may be perpendicular to a connection surface of the base 20. The connection surface of the base 20 is a surface on which the base 20 is connected to the first lifting component 31 and the driving component 42.

In one embodiment, the radiation detection device further includes a detector 60 and at least two installation bodies 70 arranged along the Z-axis. One of the at least two installation bodies 70 is used for installation onto the C-arm 90 (shown in FIG. 4), and another one of the at least two installation bodies 70 is connected to the detector 60 in an unspecified manner, such as by screw fixation.

In one embodiment of the present disclosure, the driving component 42 is rotatably connected to the base 20 around the Z-axis, and the rotation of the driving component 42 drives the second lifting component 41 to move along the Z-axis relative to the base 20, that is, the rotational motion of the driving component 42 can be converted into the linear motion of the second lifting component 41 along the Z-axis. Further, two of the at least two installation bodies 70, which are at least partially adjacent two each other (also referred to as two adjacent installation bodies 70), are connected by a lifting module, that is, one of the two adjacent installation bodies 70 is connected to the first lifting component 31. For example, the first lifting component 31 is provided with a connecting component 73, and the first lifting component 31 is connected to the installation body 70 through the connecting component 73. Another one of the two adjacent installation bodies 70 is connected to the second lifting component 41.

In one embodiment of the present disclosure, the first lifting component 31 and the second lifting component 41 move in opposite directions. The first lifting component 31 and the second lifting component 41 can be driven to move synchronously by a motor, thereby increasing the synchronicity and stability of the movement of the first lifting component 31 and the second lifting component 41. Of course, in some embodiments, the first lifting component 31 and the second lifting component 41 can also be driven by two separate motors, and the movement can be synchronous or asynchronous. For example, FIG. 2 shows two adjacent installation bodies 70. The two adjacent installation bodies 70 are connected by a lifting module to bring them closer to or farther away from each other.

In this configuration, the size of the entire lifting module along the Z-axis can be adjusted, the space occupied by the entire device along the Z-axis is reduced while the movement range of the detector 60 along the Z-axis remains unchanged. Specifically, adjacent installation bodies 70 are connected by the lifting module. The first lifting component 31 and the driving component 42 of the lifting module extend linearly along the Z-axis. The first lifting component 31 is movable along the Z-axis relative to the base 20, the driving component 42 is stationary along the Z-axis relative to the base 20, and the driving component 42 drives the second lifting component 41 to move along the Z-axis. The first lifting component 31 and second lifting component 41 move in opposite directions along the Z-axis, which allows the size of the entire lift module to be adjusted along the Z-axis, thereby reducing the space occupied by the radiation detection device on the Z-axis while the range of motion of the detector 60 along the Z-axis keeps unchanged. The single-stage lifting module in the related technology, to achieve 2× travel distance, the movement of the single-stage lifting module is required to complete 2× travel distance. However, for the two lifting components in the present disclosure, to achieve 2× travel distance, each lifting component only needs to complete X travel distance, reducing the space occupied by the X-radiation detection device on the Z-axis when the lifting device is in its original non-lifting state.

FIG. 4 is a schematic diagram of a medical imaging equipment of one embodiment of the present disclosure. The medical imaging equipment of the present embodiment includes a C-arm 90, a radiation source 100, and the aforementioned radiation detection device. With regard to the C-arm 90, the radiation source 100, and the detector 60, those skilled in the art can understand them by referring to the description in FIG. 1, which is not be elaborated here. Further, the C-arm 90 has two ends radially opposite to each other. each of the two ends of the C-arm 90 has an inner arc surface. The radiation source 100 is installed on the inner arc surface of one end, and the detector 60 is installed on the inner arc surface of the other end. Specifically, one of the at least two installation bodies 70, without installing the detector 60, is connected to the inner arc surface of the other end of the C-arm 90. After the radiation detection device of the present embodiment is applied to the medical imaging equipment, due to the adjustable size of the radiation detection device along the Z-axis, it can ensure a sufficiently large range of SID while the overall structural size of the device being too large is avoided. When the medical examination is completed, the first lifting component 31 in the lifting module moves close to the second lifting component 41 to reduce the overall size of the device.

It should be noted that when there are two installation bodies 70, one installation body 70 is connected to the first lifting component 31 and the other installation body 70 is connected to the second lifting component 41 as described earlier. When there are at least three installation bodies 70, two adjacent installation bodies 70 can be connected by the lifting module, and another two adjacent installation bodies 70 can be connected by other structures to ensure that the two adjacent installation bodies 70 are relatively stationary. For example, the radiation detection device includes three installation body 70 arranged along the z-axis, denoted as a first installation body, a second installation body, and a third installation body that are adjacent in sequence. The first installation body and the second installation body can be connected by a lifting module, and the second installation body and the third installation body can be connected by another lifting module. It is also possible that the first installation body and second installation body are connected by other structures to keep them relatively stationary, and the second installation body and third installation body are connected by the lifting module. It is understandable that the detector 60 can be attached to any of the first installation body, second installation body, and third installation body. For the purpose of optimizing the structure, it is preferred in this embodiment that the count of the at least two installation bodies 70 is two and they are connected to each other via the lifting module. Of course, a person skilled in the art may also configure multiple installation bodies 70 and at least two corresponding lifting modules based on the relationship that two adjacent installation bodies 70 are connected by one lifting module. In one of the embodiments, when the count of the at least two installation bodies 70 is at least three and the count of corresponding lifting modules is at least two, the arrangement direction of the at least two lifting modules is along the z-axis.

It should be known that when the radiation detection device is installed on the C-arm 90, the direction of the Z-axis is approximately in the direction of the line connecting the two ends of the C-arm 90. “approximately” used herein means that an included angle between the direction of the Z-axis and the direction of the line connecting the two ends of the C-arm 90 is smaller than a specific degree, such as less than 5°, 3°, 1°. For example, the direction of the Z-axis is the direction of the line connecting the two ends of the C-arm 90. In one of the embodiments, the direction of the Z-axis is the direction of the line connecting the two ends of the C-arm 90. The direction of the Z-axis varies depending on the application scenarios of the C-arm 90. For example, when the C-arm 90 is in a vertical position, the plane defined by the C-arm 90 is perpendicular to the horizontal plane, at this time the Z-axis is perpendicular to the horizontal plane. As another example, when the C-arm 90 is in a horizontal position, the plane defined by the C-arm 90 is parallel to the horizontal plane, in which case the Z-axis is parallel to the horizontal plane direction.

Continue to refer to FIG. 1, the existing medical imaging equipment uses a single-stage lifting structure 10. The size of the single-stage lifting structure is fixed. To ensure that the detector 60 has sufficient lifting range to meet the SID, the lifting structure 10 needs more space in the lifting direction to ensure that the detector 60 has sufficient travel range. Generally, the lifting structure 10 needs to be installed on the end face of the C-arm (here referring to the end face of the C-arm 90 along its own circumferent direction, that is, the end face perpendicular to the arc direction of the C-arm 90, not the inner arc surface of one end of the C-arm 90) to ensure that there is sufficient distance between the radiation source 100 and the imaging plane of the detector 60. The C-arm 90 can perform a slip ring motion driven by other related driving devices of the medical imaging equipment, such as robots. The slip ring motion can be understood as a circular motion of the C-arm 90 along its own arc direction. To meet the clinical requirements, the C-arm 90 must have a travel range of at least 180°for the circular motion, which requires that an arc length of the C-arm 90 is at least ½ of the circular length. For medical imaging equipment using a single-stage lift structure, since the single-stage lift structure is installed on one end of the C-arm in the arc direction, the arc length needs to be extended from the other end of the C-arm 90 (the end where the radiation source 100 is installed) to ensure that the total arc length of the C-arm 90 meets the 180°angle requirement. This would cause the end of the C-arm 90, where the radiation source 100 is installed, to protrude outward (shown at A in FIG. 1), which would not only be detrimental to the overall aesthetics but also have an impact on the slip ring movement of the C-arm 90 and the movement posture of the C-arm 90. For example, when the C-arm 90 is in a vertical position and makes circular motion around the vertical direction, the protruding part at A is prone to bumping into nearby objects or even medical staff.

Refer to FIG. 4, the radiation detection device of the embodiment of the present disclosure is adjustable in size along the Z-axis. The radiation detection device can be installed on the inner arc surface at one end of the C-arm 90, and the radiation source 100 is installed on the inner arc surface at the other end of the C-arm 90. Compared with medical imaging equipment using a single-stage lifting structure, for a medical imaging equipment including the radiation detection device of the present disclosure, on the one hand, the end of the C-arm 90 where the detector 60 is installed has a longer arc length, and on the other hand, the arc length of the other end of the C-arm 90 where the radiation source 100 is installed do not need to be extended to meet the travel requirements of the slip ring movement. Thus, the protruding part at A of the end of the C-arm 90 where the radiation source 100 is installed in FIG. 1 is solved, thereby reducing the risk of the end of the C-arm 90 where the radiation source 100 is installed colliding with medical personnel or objects and enhancing the safety factor of the medical imaging equipment.

In one embodiment of the present disclosure, referring to FIG. 3, the second lifting component 41 and the driving component 42 are eccentrically set relative to the Z-axis, that is, the center of a section, perpendicular to the Z-axis, of the second lifting component 41 and the center of a section, perpendicular to the Z-axis, of the driving component 42 do not coincide. The section of the second lifting component 41 perpendicular to the Z-axis partially overlaps with the section of the driving component 42 perpendicular to the Z-axis. In this way, a certain space can be provided between the second lifting component 41 and the driving component 42 for wiring, so that cable can be hidden in the second lifting unit 40, preventing the cable from being exposed to the outside, further protecting the cable, extending its service life, and also enhancing its aesthetic appeal. In some other embodiments, the second lifting component 41 and the driving component 42 are concentrically arranged relative to the Z-axis, the driving component 42 is tubular, and the driving component 42 is at least partially located in the second lifting component 41, that is, the second lifting component 41 and the driving component 42 are coaxially arranged, the driving component 42 is tubular, and the driving component 42 is arranged movably through the second lifting component 41, The second lifting component 41 is thus driven to move along the Z-axis relative to the base 20.

Further, the first lifting unit 30 further includes a reference component 32 connected to the first lifting component 31. The reference component 32 is installed on the base 20 and is set to be rotatable around its own axis. The first lifting unit 30 is configured to convert a rotational motion of the reference component 32 into a linear motion of the first lifting component 31 along the Z-axis. In some other embodiments, the radiation detection device also includes a linear drive motor directly connected to the first lifting component 31 to drive the first lifting component 31 to move along the Z-axis.

In one embodiment, the first lifting unit 30 can be configured based on the principle of the ball screw. Specifically, at least one segment of the first lifting component 31 along the axial direction is provided with an external thread so that the at least one segment of the first lifting component 31 is configured as a first lead screw, and the reference component 32 is rotatably installed on the base 20, with the axis direction of the reference component 32 being the same as the Z-axis direction, and the reference component 32 is threaded to the first lead screw. Thus, the reference component 32 has a nut feature, the first lifting component 31 has a lead screw feature, and the reference component 32 rotates to drive the first lifting component 31 to move along the Z-axis. In one of the embodiments, the reference component 32 is a nut.

In an alternative embodiment, the first lifting unit 30 may be configured based on the principle of rack and gear. Specifically, at least a part of the first lifting component 31 along the axial direction is configured as a rack. The reference component 32 is rotatably installed on the base 20. The axis direction of the reference component 32 is the same as the Z-axis direction. The reference component 32 has gear characteristics to achieve the meshing connection between the reference component 32 and the rack. In one of the embodiments, the reference component 32 is a gear.

Similarly, a second lifting unit 40 can be configured based on the principle of ball screw. Specifically, at least one segment of the driving component 42 along the axial direction has an external thread so that the at least one segment of the driving component 42 is configured as a second lead screw. The second lifting component 41 has a nut feature. The second lifting component 41 is threaded to the second lead screw. In one of the embodiments, the second lifting component 41 includes a first part 411 and a second part 412 connected to each other (which, for example, can be detachable by a screw). The first part 411 has physical contact with the driving component 42. The first part 411 is mechanically connected to the driving component 42, for example, by a threaded connection. And/or the second part 412 is mechanically connected to the driving component 42, for example via a threaded connection. The second part 412 is connected to the installation body 70. The first part 411 is driven by the driving component 42 to move along the Z-axis, thereby moving the second part 412 and then moving the installation body 70 relative to the base 20.

In one embodiment of the present disclosure, the lifting module further includes a driving unit 50. The driving unit 50 is respectively connected to the reference component 32 and the driving component 42. The driving unit 50 is used to drive the reference component 32 and the driving component 42 to rotate synchronously, thereby driving the first lifting component 31 and the second lifting component 41 to move synchronously along the z-axis and to move synchronously towards each other or synchronously against each other. In one embodiment, the driving unit 50 includes a first transmission structure (not illustrated), a second transmission structure (not illustrated), a first gear 51, and a second gear 52. The first gear 51 and the second gear 52 are coaxially fixed along the Z-axis and can rotate synchronously around the Z-axis. One of the first gear 51 and the second gear 52 meshes with the first transmission structure, and the first transmission structure meshes with the driving component 42. Specifically, a third gear 53 is coaxially fixed on the driving component 42, and the first transmission structure meshes with the third gear 53. The other one of the first gear 51 and the second gear 52 is meshed with the second transmission structure. The second transmission structure is meshed with the reference component 32. Specifically, the reference component 32 is coaxially fixed with a fourth gear 54, and the second transmission structure is meshed with the fourth gear 54. The mechanical characteristics of the first transmission structure and the second transmission structure are not limited, and a person skilled in the art may configure them according to the actual situation. For example, the first transmission structure and the second transmission structure may be a synchronous transmission belt. The synchronous transmission belt connects the first gear 51 and the third gear 53, or connects the second gear 52 and the fourth gear 54. The first transmission structure and the second transmission structure can be a combination of multiple gear parts to achieve the transmission of motion relations. It is understandable that when the first lifting unit 30 is configured based on the principle of rack and gear, the second transmission structure can take the form of a combination of multiple gear pieces, some of the multiple teeth gear pieces are in the form of bevel gear fits.

As details of further embodiments, the driving unit 50 also includes a rotating motor 55, a coupling 57, and an installation shaft 56. The installation shaft 56 is axially aligned with the Z-axis direction. The rotating motor 55 is set on the base 20 through a motor installation seat 58. The installation shaft 56 is connected to an output shaft of the rotating motor 55 through the coupling 57. The first gear 51 and the second gear 52 are coaxially fixed on the installation shaft 56. The rotating motor 55 drives the installation shaft 56 to rotate, thereby driving the first gear 51 and the second gear 52 to rotate synchronously.

In the specific implementation of the present disclosure, when the upper program controls the rotating motor 55 to rotate in one direction, a nut connected to the first lifting component 31 and a lead screw connected to the second lifting component 41 are driven to rotate through a synchronous transmission belt. The first lifting component 31 and its installation bod(ies) 70, and the second lifting component 41 and its installation bod(ies) 70, move in opposite directions relative to the intermediate body 80. When the lifting module is fixed to the C-arm 90, the above movement is manifested as the contraction movement of the entire lifting module, corresponding to an increase in SID. When the upper program controls the rotating motor 55 to rotate in the opposite direction, the synchronous transmission belt drives the nut connected to the first lifting component 31 to rotate and the lead screw connected to the second lifting component 41 to rotate in the opposite direction, enabling the first lifting component 31 and its installation bod(ies) 70 and the second lifting component 41 and its installation bod(ies) 70 to move opposite to the intermediate body 80. When the lifting module is fixed to the C-arm 90, the above movement is manifested as an unfolding movement of the entire lifting module, corresponding to the reduction of SID.

In one embodiment of the present disclosure, a moving rate of the first lifting component 31 is equal to a moving rate of the second lifting component 41, thereby achieving the equal moving rates of the two connected installation bodies 70. In some other embodiments, the moving rates of the first lifting component 31 and the second lifting component 41 may also be unequal.

For example, the first lifting unit 30 and the second lifting unit 40 are configured by the principle of ball screw, so the axis direction of the reference component 32 is the same as the Z-axis direction. The first gear 51 is fitted with the fourth gear 54, the second gear 52 is fitted with the third gear 53. A count of teeth of the first gear 51 is a, the second gear 52 has b, the third gear 53 has c, the fourth gear 54 has d, the lead of the first lifting component 31 is e, and the lead of the driving component 42 is f, If the above conditions are met, d×b×f=c×a×e, the moving speeds of the first lifting component 31 and the second lifting component 41 are equal. It is understandable that the lead of the first lifting component 31 refers to a distance that the first lifting component 31 moves after the fourth gear 54 drives the reference component 32 to rotate one turn, and the lead of the second lifting component 41 refers to a distance that the second lifting component 41 moves after the third gear 53 drives the driving component 42 to rotate one turn.

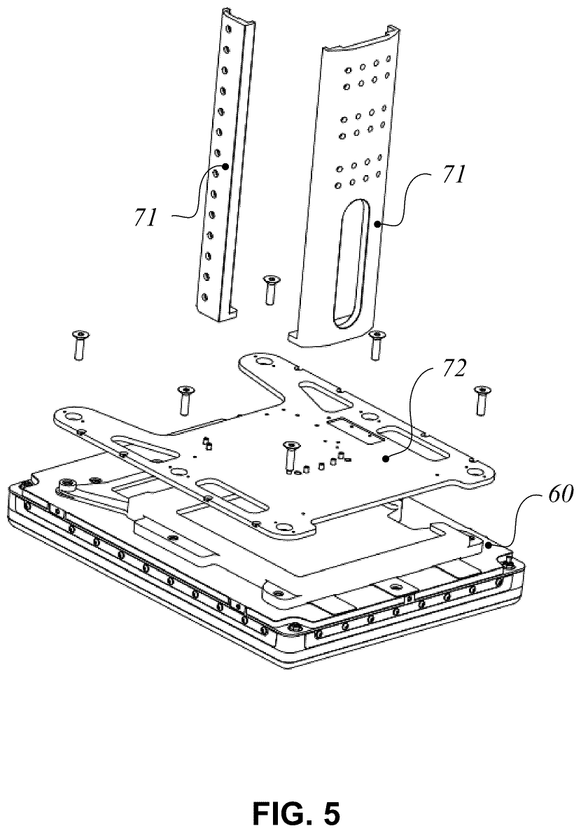

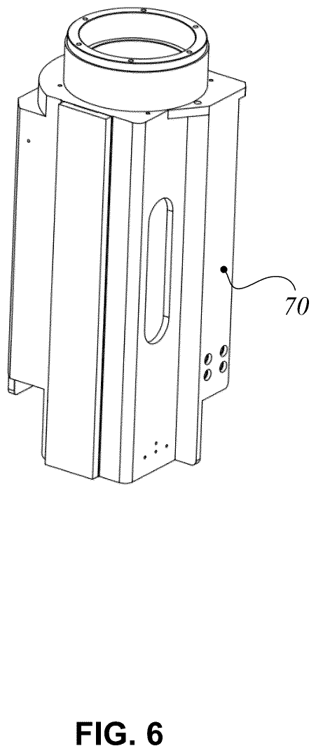

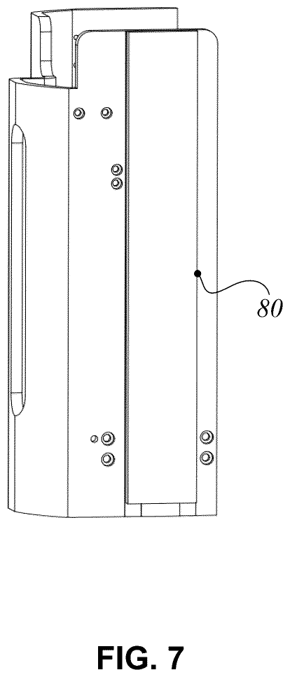

FIG. 5 is an explosion diagram of the detector and installation body of one embodiment of the present disclosure, FIG. 6 is a schematic diagram of the installation body 70 of one embodiment of the present disclosure, and FIG. 7 is a schematic diagram of the intermediate body 80 of one embodiment of the present disclosure. The installation body 70 is connected to the detector 60 by an installation substrate 72. The installation body 70 is cylindrical around the z-axis (as shown in FIG. 6), or the installation body 70 may also be shell-like around the Z-axis. For example, the installation body 70 shown in FIG. 5 includes at least two connecting substrates 71 arranged around the Z-axis so that the installation body 70 is cylindrical or shell-like around the Z-axis. Refer to FIG. 7, the radiation detection device further includes an intermediate body 80 The intermediate body 80 is cylindrical or shell-shaped around the z-axis, and the base 20 is fixed in the intermediate body 80. In combination with refer to FIG. 2, the intermediate body 80 is movably lapped with two adjacent installation bodies 70 along the Z-axis, respectively. That is, the intermediate body 80 has lapping edges with the installation bodies 70 on either side of the intermediate body 80, and the intermediate body 80 fits with the installation bodies 70 to cover the lifting module. In addition, a guiding structure is provided between the intermediate body 80 and the installation body 70 to enable the movement and guidance of the intermediate body 80 and the installation body 70 along the Z-axis. The guiding structure may be in the form of a guiding groove, a guiding plate, a guide rail, etc. In such a configuration, the lifting module is covered inside, allowing the internal structure to be covered inside and not to be exposed to protect the internal structure and enhance the safety of the radiation detection device.

Based on the aforementioned radiation detection device, an embodiment also provides a medical imaging equipment. For example, a digital subtraction angiography device, including the aforementioned radiation detection device, the C-arm 90 and the radiation source 100 (as shown in FIG. 4). The C-arm 90 has two ends that are radially opposite each other, and each of the two ends of the C-arm 90 have inner arcs, with the radiation source 100 installed on the inner arcs surface at of one end of the C-arm 90. Another one of the at least two adjacent installation bodies 70 in the radiation detection device that is not connected to the detector 60 is connected to the inner arc surface of the other end of the C-arm 90. The radiation source 100 emits rays (such as X-rays), which can be received by the detector 60 after passing through the patient for operations such as medical imaging to assist applications in fields such as angiography, cardiology, and neurology.

In one embodiment of the present disclosure, a medical imaging equipment is also provided, including: a gantry, a radiation source 100 and a radiation detection device. The radiation detection device includes a lifting module and a detector 60. The gantry has two ends opposite each other. The radiation source 100 is configured at one end of the gantry. The lifting module includes the first lifting unit 30 and the second lifting unit 40. The detector 60 is arranged at the other end of the gantry through the lifting module. The first lifting unit 30 and the second lifting unit 40 are arranged perpendicularly to the detector 60 in parallel. The first lifting unit 30 and the second lifting unit 40 are configured to drive the detector 60 to move near or away from the radiation source 100. Specifically, the gantry is a C-arm 90 having two ends opposite each other, and both ends of the C-arm 90 have an inner arc surface. The radiation source 100 is arranged on the inner arc surface of one end of the C-arm 90, and the detector 60 is arranged on the inner arc surface of the other end of the C-arm 90 through the lifting module.

In another embodiment of the present disclosure, the lifting module further comprises a base 20. The first lifting unit 30 includes a first lifting component 31 extending along the Z-axis. The first lifting component is movably connected to the base 20 along the Z-axis, wherein the Z-axis is parallel to the direction of movement of the detector 60. The second lifting unit 40 includes a driving component 42 extending along the Z-axis and a second lifting component 41 connected to the driving component 42. The driving component 42, is configured to drive the second lifting component 41 to move along the Z-axis.

In summary, in the radiation detection device and medical imaging equipment provided by the present disclosure, the radiation detection device includes at least one lifting module, a detector 60 and at least two installation bodies 70 arranged along the Z-axis, and the lifting module includes a base 20, a first lifting unit 30, and a second lifting unit 40; The first lifting unit 30 includes a first lifting component 31 extending along the Z-axis, which is movably connected to the base 20 along the Z-axis. The second lifting unit 40 includes a driving component 42 extending along the Z-axis and a second lifting component 41 connected to the driving component 42. The driving component 42 is rotatably connected to the base 20 around the Z-axis, and the rotation of the drive unit drives the second lifting component 41 to move along the Z-axis; At least two adjacent installation bodies 70, one of the two adjacent installation bodies 70 of the at least two installation bodies 70 is connected to the first lifting component 31 and the other of the two adjacent installation bodies 70 to the second lifting component 41; The first lifting component 31 and the second lifting component 41 move in opposite directions. one of the two adjacent installation bodies 70 of the at least two installation bodies 70 is connected to the detector 60; and, the other of the two adjacent installation bodies 70 is connected to the C-arm 90. In such a configuration. The two adjacent installation bodies 70 are connected by lifting modules, the first lifting component 31 and the second lifting component 41 of the lifting modules extend linearly along the z-axis, the first lifting component 31 is movable along the Z-axis relative to the base 20, the driving component 42 is stationary along the Z-axis relative to the base 20, and the driving component 42 drives the second lifting component 41 along the z-axis. And the first lifting component 31 and the second lifting component 41 move in opposite directions along the Z-axis, which allows the size of the entire lift module to be adjusted along the Z-axis, thereby reducing the space occupied by the ray detector on the Z-axis while keeping the range of motion of the detector 60 along the Z-axis unchanged.

The above description is only a description of the preferred embodiment of the present disclosure and is not any limitation of the scope of the present disclosure. Any changes or modifications made by ordinary technicians in the field of the present disclosure based on the above disclosure are within the protection scope of the technical solution of the present disclosure.

1. A radiation detection device including a lifting module, wherein
the lifting module includes a base, a first lifting unit, and a second lifting unit;
the first lifting unit includes a first lifting component extending along a Z-axis, and the first lifting component is movably connected to the base along the Z-axis;
the second lifting unit includes a driving component extending along the Z-axis and a second lifting component connected to the driving component, and the driving component is configured to drive the second lifting component to move along the Z-axis; and
a direction of the Z-axis is perpendicular to a connection surface of the base, wherein the connection surface of the base is a surface on which the base is connected to the first lifting component and the driving component.

2. The radiation detection device of claim 1, wherein:
the radiation detection device further includes a detector and at least two installation bodies arranged along the Z-axis;
one of at least two adjacent installation bodies of the at least two installation bodies is connected to the first lifting component and another one of the at least two adjacent installation bodies is connected to the second lifting component; and
the detector is connected to one of the at least two adjacent installation bodies of the at least two installation bodies.

3. The radiation detection device of claim 1, wherein:
the second lifting component and the driving component are eccentrically arranged relative to the z-axis, and
a section of the second lifting component perpendicular to the z-axis is partially overlapped with a section of the driving component perpendicular to the z-axis.

4. The radiation detection device of claim 1, wherein:
the first lifting unit further includes a reference component connected to the first lifting component,
the reference component is configured to be rotatable around its own axis, and
the first lifting unit is configured to convert a rotational motion of the reference component into a linear motion of the first lifting component along the z-axis.

5. The radiation detection device of claim 4, wherein:
at least one segment of the first lifting component is configured as a first lead screw, and the reference component is threaded to the first lead screw; or
at least one segment of the first lifting component is configured as a rack, and the reference component meshes with the rack.

6. The radiation detection device of claim 1, wherein:
the driving component is configured to be rotatably connected with the base around the z-axis, and
a rotation of the driving component drives the second lifting component to move along the Z-axis.

7. The radiation detection device of claim 4, wherein:
the lifting module further includes a driving unit,
the driving unit is respectively connected to the reference component and the driving component, and
the driving unit is configured to drive the reference component and the driving component to rotate synchronously.

8. The radiation detection device of claim 4, wherein:
the driving unit includes a first transmission structure, a second transmission structure, a first gear, and a second gear;
the first gear and the second gear are coaxially fixed along the Z-axis and can rotate synchronously around the Z-axis;
one of the first gear and the second gear meshes with the first transmission structure, and the first transmission structure meshes with the driving component; and
another one of the first gear and the second gear meshes with the second transmission structure, and the second transmission structure meshes with the reference component.

9. The radiation detection device of claim 1, wherein:
at least one segment of the driving component is configured as a second lead screw, and the second lifting component is threaded with the second lead screw.

10. The radiation detection device of claim 1, wherein:
moving rates of the first lifting component and the second lifting component are equal.

11. The radiation detection device of claim 2, the radiation detection device further including an intermediate body, wherein:
the intermediate body and one of the at least two installation bodies each have a cylindrical or shell-like shape around the z-axis,
the base is fixed within the intermediate body,
the intermediate body is movably lapped with at least two adjacent installation bodies of the at least two installation bodies along the Z-axis respectively, and
the intermediate body is in conjunction with the at least two adjacent installation bodies to cover the lifting module.

12. (canceled)

13. The radiation detection device of claim 1, wherein:
the second lifting component and the driving component are concentric with respect to the Z-axis;
the driving component has a tubular structure; and
the driving component is configured to be movably inserted through the second lifting component.

14. The radiation detection device of claim 1, wherein:
the second lifting component includes a first part and a second part connected to each other;
the first part is mechanically connected to the driving component by thread;
the second part is mechanically connected to the driving component;
the second part is connected to another one of the at least two adjacent installation bodies; and
the first part is configured to be driven by the driving component to move along the Z-axis.

15. (canceled)

16. The radiation detection device of claim 7, the driving unit further including a rotating motor, a coupling, and an installation shaft, wherein:
an axial direction of the installation shaft is the same as a direction of the Z-axis;
the rotating motor is set on the base through a motor installation seat;
the installation shaft is connected to an output shaft of the rotating motor through the coupling; and
the first gear and the second gear are coaxially fixed on the installation shaft.

17. The radiation detection device of claim 11, wherein:
a guiding structure is provided between the intermediate body and at least part of the at least two installation bodies;
the guiding structure includes a guiding groove, a guiding plate, or a guiding rail.

18. (canceled)

19. The radiation detection device of claim 5, wherein:
at least one segment of the first lifting component is configured as a first lead screw, and the reference component is a nut; and
the at least one segment of the first lifting component is configured as a rack, and the reference component is configured as a gear.

20. The radiation detection device of claim 1, wherein:
the first lifting component and the second lifting component move in opposite directions relative to the base.

21. A medical imaging equipment including:
a C-arm, having two ends opposite each other, wherein each of the two ends of the C-arm have an inner arc surface;
a radiation source installed on an inner arc surface at one end of the C-arm; and
the radiation detection device of claim 2, wherein in the radiation detection device, another one of the at least two adjacent installation bodies not connected to the detector is connected to an inner arc surface of another end of the C-arm.

22. (canceled)

23. A medical imaging equipment including:
a gantry having two ends opposite each other;
a radiation source configured at one end of the gantry;
a radiation detection device including:
a lifting module including a first lifting unit and a second lifting unit; and
a detector arranged at another end of the gantry through the lifting module, wherein
the first lifting unit and the second lifting unit are arranged perpendicularly to the detector in parallel;
the first lifting unit and the second lifting unit are configured to drive the detector to move near or away from the radiation source.

24. The medical imaging equipment of claim 23, wherein:
the gantry is a C-arm having two ends opposite each other, and both ends of the C-arm have an inner arc surface;
the radiation source is arranged on the inner arc surface at one end of the C-arm; and
the detector is arranged on the inner arc surface of the other end of the C-arm through the lifting module.

25-31. (canceled)
